(19) United States
(12) Patent Application Publication
(10) Pub. No.: US 2021/0235673 A1
(43) Pub. Date: Aug. 5, 2021

(54) GRAVES' OPHTHALMOPATHY PHENOTYPE ANIMAL MODEL, CONSTRUCTION METHOD THEREFOR, AND METHOD FOR SCREENING THERAPEUTIC MATERIAL FOR GRAVES' OPHTHALMOPATHY

(71) Applicant: SAMSUNG LIFE PUBLIC WELFARE FOUNDATION, Seoul (KR)

(72) Inventors: Dong Hui LIM, Seoul (KR); Jae Ryung KIM, Seongnam-Si Gyeonggi-do (KR); Dae Young PARK, Jeju-Si Jeju-do (KR); Tae Young CHUNG, Seongnam-Si Gyeonggi-do (KR)

(21) Appl. No.: 16/972,048

(22) PCT Filed: Jun. 5, 2019
(86) PCT No.: PCT/KR2019/006788

§ 371 Date: Dec. 4, 2020

(30) Foreign Application Priority Data
Jun. 5, 2018 (KR) 10-2018-0064752

(51) Int. Cl. A01K 67/027; A61K 49/00

(52) U.S. Cl. CPC A01K 67/027; A61K 49/0008; A01K 2227/105; A01K 2207/20; A01K 2267/0325

(57) ABSTRACT

The present disclosure relates to a method for preparing a Graves' ophthalmopathy phenotype animal model, the method including a step of administering zymosan A to a subject other than humans, a Graves' ophthalmopathy phenotype animal model prepared thereby, and a method for screening a therapeutic material for alleviation or treatment of Graves' ophthalmopathy. By using the method for preparing a Graves' ophthalmopathy phenotype animal model, which includes a step of administering zymosan A to a subject other than humans according to the present disclosure, an experimental animal model for Graves' ophthalmopathy, which simultaneously exhibits blepharitis, orbital tissue inflammation, and exophthalmos, may be obtained. In addition, the animal model prepared by the preparation method of the present disclosure may be advantageously used for researching the development of a therapeutic agent for Graves' ophthalmopathy the etiology of which has not been yet accurately revealed.

### TECHNICAL FIELD

The present disclosure relates to a method for preparing a Graves' ophthalmopathy phenotype animal model, the method including administering zymosan A to a non-human subject, a Graves' ophthalmopathy phenotype animal model prepared by the above preparation method, a composition for preparing a Graves' ophthalmopathy phenotype animal model, the composition containing zymosan A as an active ingredient, and a method for screening a substance for alleviation or treatment of Graves' ophthalmopathy.

### BACKGROUND ART

Graves' ophthalmopathy is an orbital disease that occurs in association with thyroid disease, and has been referred to as thyroid-associated ophthalmopathy. Graves' ophthalmopathy has clinical symptoms such as exophthalmos, eyelid retraction, restrictive myopathy, and compressive optic neuropathy due to orbital tissue inflammation, edema, and adipogenesis, enlargement and fibrosis of the extraocular muscle due to the humoral and cellular immune response caused by thyroid disease. Graves' ophthalmopathy is generally associated with hyperthyroidism, or is associated with normal thyroid function and hypothyroidism. Graves' ophthalmopathy is associated with sex hormones and has a higher prevalence in women than in men. Although the etiology thereof is still not well understood, the etiology of the Graves' ophthalmopathy is considered to be an autoimmune mechanism. Most of the patients suffer from mild symptoms, but 10 to 15% of patients suffer from severe forms of thyroid orbitopathy such as exophthalmos, restrictive myopathy, and compressive optic neuropathy.

Graves' ophthalmopathy has been mainly understood as a disease of orbital tissue caused by autoimmunity. In almost all patients thereof, treatment for an acute phase includes administration of corticosteroid agents or radiotherapy. Orbital decompression surgery is performed when necessary after active inflammation is stabilized. This is the only time when orbital tissues beyond the acute stage of disease may be obtained. Thus, there is a limit to research using human tissues.

An approach for researching treatment methods for diseases such as human Graves' ophthalmopathy includes a method for preparing and using experimental disease animal models. The method for preparing the experimental disease animal model includes a method for preparing a transgenic animal model using genetic recombination.

Thus, establishing a mouse model representing the phenotype of Graves' ophthalmopathy is a first step in treating Graves' ophthalmopathy. Accordingly, animal models that reflect Graves' ophthalmopathy have been tried continuously. However, there is no suitable model that is widely used yet.

### SUMMARY

### Technical Purpose

Accordingly, the present inventors have studied experimental disease animal models to study treatment methods for diseases such as Graves' ophthalmopathy and then have identified that Graves' ophthalmopathy phenotype occurred in SKG mice treated with zymosan A at a specific concentration. In this way, the present disclosure has been completed.

Therefore, a purpose of the present disclosure is to provide a method for preparing a Graves' ophthalmopathy phenotype animal model, the method including a step of administering zymosan A to a non-human subject, the Graves' ophthalmopathy phenotype animal model prepared by the above preparation method, a composition for preparing the Graves' ophthalmopathy phenotype animal model, the composition containing zymosan A as an active ingredient, and a method for screening a substance for alleviation or treatment of Graves' ophthalmopathy, the method including (a) administering a candidate substance for an alleviation or treatment agent for the Graves' ophthalmopathy phenotype into the animal model prepared by the preparation method, and (b) identifying an alleviation or treatment effect of the Graves' ophthalmopathy phenotype in the animal model into which the candidate substance is administered, compared to a control into which the candidate substance is not administered.

### Technical Solution

In order to achieve the above purpose, the present disclosure provides a method for preparing a Graves' ophthalmopathy phenotype animal model, the method including a step of administering zymosan A to a subject other than humans

Further, the present disclosure provides a Graves' ophthalmopathy phenotype animal model prepared by the above preparation method.

Further, the present disclosure provides a composition for preparing an animal model of Graves' ophthalmopathy phenotype, the composition containing zymosan A as an active ingredient.

Further, the present disclosure provides a method for screening a substance for alleviation or treatment of Graves' ophthalmopathy, the method including (a) administering a candidate substance for an alleviation or treatment agent for Graves' ophthalmopathy phenotype into the animal model prepared by the preparation method, and (b) identifying an alleviation or treatment effect of the Graves' ophthalmopathy phenotype in the animal model into which the candidate substance is administered, compared to a control into which the candidate substance is not administered.

### Advantageous Effects

When using the Graves' ophthalmopathy phenotype animal model preparation method according to the present disclosure which includes the step of administering zymosan A to a subject other than humans, it is possible to obtain a Graves' ophthalmopathy experimental animal model that may simultaneously represent blepharitis and exophthalmos. In addition, the animal model prepared by the preparation method according to the present disclosure may be usefully used in research for development of a therapeutic agent for Graves' ophthalmopathy whose etiology has not yet been accurately identified.

### BRIEF DESCRIPTION OF DRAWINGS

FIG. 1 is a schematic diagram of the overall experimental execution process according to the present disclosure in which Graves' ophthalmopathy animal model preparation and histological visual analysis of the model are performed.

FIG. 2 is a diagram showing the result of comparing an orbital part of the animal model prepared by administering zymosan A to SKG mice and an orbital part of a control group not treated with zymosan A with each other.

FIG. 3 shows the results of the eyelid thickness and meibomian gland thickness of SKG mice treated with zymosan A, compared to those of the control not treated with zymosan A (FIG. 3A) and the results of quantitative graphs thereof (FIG. 3B, FIG. 3C), the result of identifying the inflammatory cells around the orbit (FIG. 3D) and the result of a quantitative graph thereof (FIG. 3E). (*p value<0.05).

FIG. 4 shows the results of magnetic resonance imaging of SKG mice before and after zymosan A treatment compared to the zymosan A non-treated control (FIG. 4A), and a quantitative graph thereof (FIG. 4B). (*p value<0.05).

FIG. 5 shows the results of the orbital adipose tissue surrounding the optic nerve and the inflammatory cells infiltrating the orbit of adult SKG mice on 3 months after administration of zymosan A thereto (FIG. 5A), and the result of quantitative graphs thereof (FIG. 5B, FIG. 5C). (*p value<0.05).

FIG. 6 shows the result of identifying the distribution of UCP-1 positive beige adipocytes (FIG. 6A) and the result of a quantitative graph thereof (FIG. 6B). (*p value<0.05).

FIG. 7 shows the result of a quantitative comparison of adipokine expressed in orbital tissue. (*p value<0.05).

FIG. 8 shows the result of a quantitative comparison of serum cytokines. (*p value<0.05).

### DETAILED DESCRIPTIONS

The present disclosure provides a method for the preparation of a Graves' ophthalmopathy phenotype animal model, the method including administering zymosan A to a non-human subject.

Hereinafter, the present disclosure will be described in more detail.

The zymosan A used in the present disclosure is a gray-white powder of the cell wall fraction of yeast, and refers to a mixture containing polysaccharides such as glucan (58%) and mannan (18%), proteins, chitin, glycolipids and ash.

The “animal model” used in the present disclosure refers to an animal with a disease very similar to a human disease. In the study of human disease, the meaning of disease model animals is based on the physiological or genetic similarity between humans and animals. In disease research, biomedical disease model animals provide materials for research on various causes, pathogenesis and diagnosis of diseases, and allow discovering genes related to diseases through research of disease model animals, and allow understanding the interaction between genes. Further, using the model, basic data used to determine the possibility of practical use through the actual efficacy and toxicity tests of the developed new drug candidate substance may be obtained.

Further, the ‘Graves’ ophthalmopathy phenotype animal model used in the present disclosure refers to a disease having clinical symptoms such as exophthalmos, eyelid retraction, restrictive myopathy, and optic neuropathy due to orbital tissue inflammation, edema, and adipogenesis, enlargement of the extraocular muscle due to the humoral and cellular immune response caused by thyroid disease.

The term “administration” in the present disclosure means introducing a given substance to a subject in an appropriate way. The administration route of zymosan A may include any general route as long as the zymosan A may reach the target tissue through the route. The administration may include intraperitoneal administration, intravenous administration, intramuscular administration, subcutaneous administration, intradermal administration, oral administration, topical administration, intranasal administration, intrapulmonary administration, rectal administration. In the present disclosure, intraperitoneal administration was performed in a preferred example, but the present disclosure is not limited thereto.

In the present disclosure, in order to prepare an animal model with Graves' ophthalmopathy, zymosan A may be administered to the animal model in a 0.5 mg to 10 mg, preferably 1 mg to 5 mg, more preferably, 3 mg.

The term “beige fat” in the present disclosure has the same embryological origin as white fat cells stimulated by the sympathetic nervous system, and refers to fat that expresses UCP-1 while being distributed in white adipose tissue. Beige fat has a smaller vacuole than white fat has, has a high cell density, and is known as an adipocyte that performs temperature regulation (thermogenesis). Further, it was recently found that conversion from white fat to beige fat may be promoted by an immune mechanism induced by T cells.

In an animal model having the Graves' ophthalmopathy phenotype as prepared by administering the zymosan A thereto, beige fat around the optic nerve is increased. In addition, in the animal model, the thickness of the entire eyelid and the thickness of the meibomian gland are increased, and at the same time, the inflammatory response is increased due to an autoimmune reaction associated with T cells by the increased beige fat, resulting in infiltration of inflammatory cells into the orbit.

The Graves' ophthalmopathy phenotype in the present disclosure may include various symptoms that appear when inducing Graves' ophthalmopathy. Preferably the symptoms may be one or more selected from the group consisting of blepharitis and exophthalmos. The blepharitis and exophthalmos may be accompanied by an increase in the thickness of the eyelid or an increase in the thickness of the meibomian gland.

The animal model having the Graves' ophthalmopathy phenotype according to the present disclosure may be a mouse, rat, rabbit, dog or guinea pig, preferably a mouse. For example, the animal model having the Graves' ophthalmopathy phenotype according to the present disclosure may be an SKG mouse in which arthritis naturally occurs due to the production of autoreactive T cells.

Further, the present disclosure provides a Graves' ophthalmopathy phenotype animal model prepared by the above preparation method.

The preparation method according to the present disclosure refers to a method for preparing a Graves' ophthalmopathy phenotype animal model, the method including the step of administering zymosan A to a subject other than humans.

In the present disclosure, in order to prepare an animal model with Graves' ophthalmopathy phenotype, zymosan A may be administered in 0.5 mg to 10 mg to the animal model. Preferably, 1 mg to 5 mg of zymosan A may be administered to the animal model. More preferably, 3 mg of zymosan A may be administered thereto.

The animal model having the Graves' ophthalmopathy phenotype prepared by administering the zymosan A as prepared in the present disclosure is characterized by increased beige fat around the optic nerve.

Further, the Graves' ophthalmopathy phenotype exhibited by the animal model prepared in the present disclosure may be at least one selected from the group consisting of blepharitis and exophthalmos. The blepharitis and exophthalmos may be accompanied by an increase in the thickness of the eyelid or an increase in the thickness of the meibomian gland.

The animal model having the Graves' ophthalmopathy phenotype as prepared by the method according to the present disclosure may be a mouse, rat, rabbit, dog or guinea pig, preferably a mouse. For example, the animal model having the Graves' ophthalmopathy phenotype as prepared by the method according to the present disclosure may be an SKG mouse in which arthritis naturally occurs due to the production of autoreactive T cells.

Further, the present disclosure provides a composition for preparing an animal model having Graves' ophthalmopathy phenotype, the composition containing zymosan A as an active ingredient.

The composition for preparing an animal model having Graves' ophthalmopathy phenotype containing zymosan A as an active ingredient according to the present disclosure may be advantageously used to prepare an animal model having Graves' ophthalmopathy phenotype in animals other than humans

Further, the present disclosure provides a method for screening a substance for alleviation or treatment of Graves' ophthalmopathy, the method including (a) administering a candidate substance for an alleviation or treatment agent for Graves' ophthalmopathy phenotype into the Graves' ophthalmopathy phenotype animal model prepared by the preparation method, and (b) identifying an alleviation or treatment effect of the Graves' ophthalmopathy phenotype in the animal model into which the candidate substance is administered, compared to a control into which the candidate substance is not administered.

In the present disclosure, “candidate substance” means a substance to be tested as an alleviation and therapeutic agent for the Graves' ophthalmopathy or Graves' ophthalmopathy phenotype. For example, the candidate substance may contain any molecules of extracts, proteins, oligopeptides, small organic molecules, polysaccharides, polynucleotides and a wide range of compounds. These candidate substances further include natural as well as synthetic substances.

In the present disclosure, “alleviation” and “treatment” refer to all actions by which the Graves' ophthalmopathy or Graves' ophthalmopathy phenotype of the animal model is alleviated or beneficially changed via administration of the candidate substance to the model.

In the present disclosure, “control” refers to a group to which any treatments or conditions are not applied to determine whether or not the results of the experiment are properly derived. Unlike the experimental group as a group set to achieve the direct purpose of the experiment, the control is a group set to make the results of the experimental group more certain. Control in the present disclosure is preferably a group of the Graves' ophthalmopathy or Graves' ophthalmopathy phenotype animal model that is not treated with the candidate substance.

In the present disclosure, the candidate substance identified as having a therapeutic or alleviation effect of the Graves' ophthalmopathy phenotype in the step (b) may be determined as a therapeutic agent for Graves' ophthalmopathy or complications thereof.

In the present disclosure, the Graves' ophthalmopathy or Graves' ophthalmopathy phenotype animal model refers to a disease model for the Graves' ophthalmopathy or Graves' ophthalmopathy phenotype. The substance obtained by the above screening method may be used in various ways including searching for therapeutic substances, identifying side effects, and developing diagnostic methods for patients with Graves' ophthalmopathy. Thus, using this animal model, alleviation or therapeutic agents for Graves' ophthalmopathy and its complications may be developed.

Redundant contents may be omitted in consideration of the complexity of the present disclosure. Terms not otherwise defined in the present disclosure have meanings commonly used in the technical field to which the present disclosure belongs.

Hereinafter, in order to help understanding the present disclosure, Examples will be described in detail. However, the following Examples are only to illustrate the content of the present disclosure, and the scope of the present disclosure is not limited to the following Examples. The Examples of the present disclosure are provided to describe the present disclosure more completely to those with average knowledge in the art.

### EXAMPLES

### Example 1. Preparation of SKG Mice in which an Immune Response was Induced with Zymosan A

SKG mice purchased from CLEA Japan were reared in an SPF facility under the Samsung Life Science Research Institute under an appropriate environment and were used for experiments. In order to induce an intrinsic immune response in SKG mice, 3 mg of zymosan A (Z4250, Sigma-Aldrich) per one mouse was intraperitoneally administered once into the abdominal cavity of 8-week-old SKG mice. Prior to all manipulations, 40 mg of ketamine and 12 mg of xylazine per kg were intramuscularly administered to the mice. All processes of animal breeding and experimentation were carried out in accordance with the guidelines approved by the Institutional Animal Ethics Review Committee (IACUC) of Samsung Medical Center.

### Example 2. Identification of Periocular Inflammation and Exophthalmos in Zymosan A-Treated SKG Mice

2.1 Histological analysis of Periocular Inflammation and Exophthalmos in Zymosan A-Treated SKG Mice

Histological analysis of the eyeball of an adult SKG mouse was performed to identify whether the mouse prepared in Example 1 above had blepharitis and exophthalmos occurred around the eye by inducing a phenotype similar to that of Graves' ophthalmopathy.

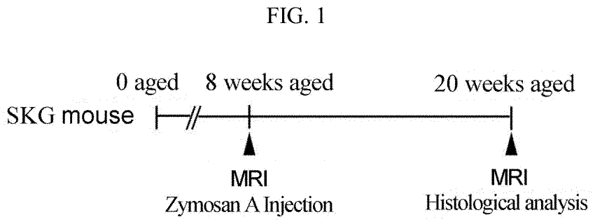

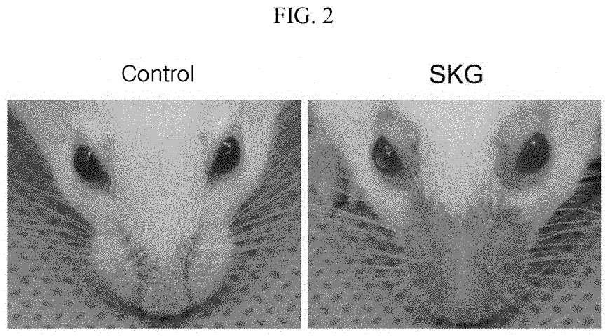

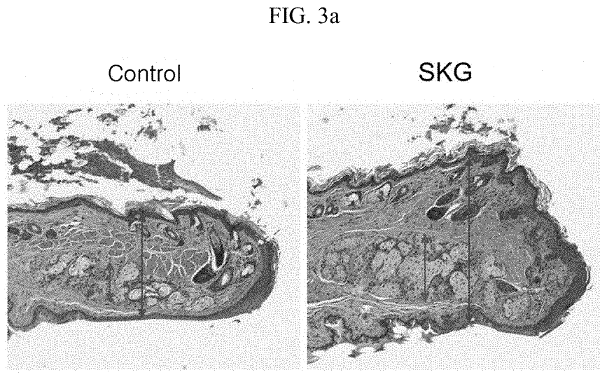

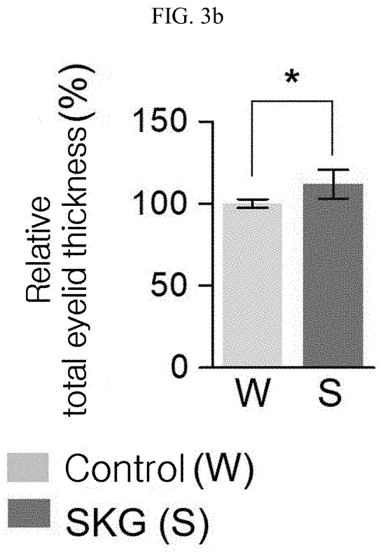

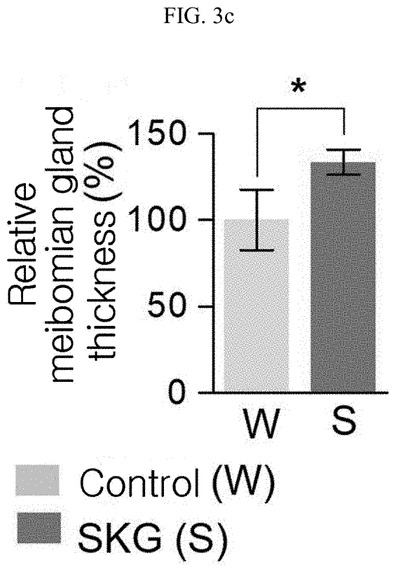

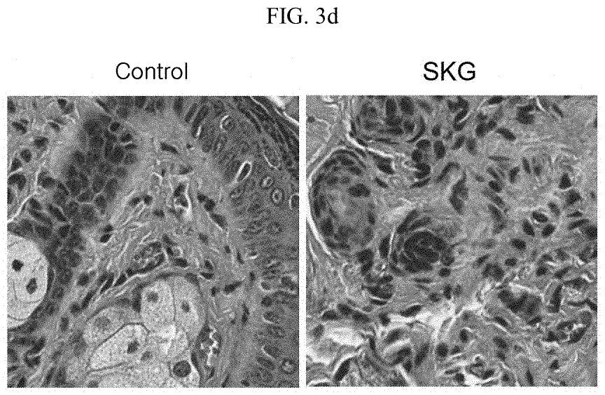

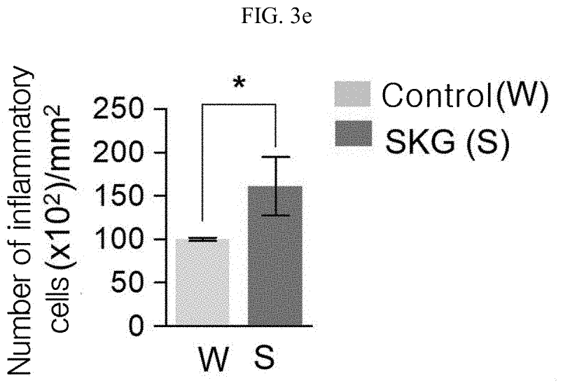

The SKG mouse prepared in Example 1 was subjected to perfusion fixation with 4% paraformaldehyde solution at 20 weeks of age, and then histological analysis was performed and magnetic resonance images were taken. The orbital tissue including the surrounding bone tissue was completely removed while the orbital tissue was not damaged. The orbital tissue was treated with EDTA for 24 hours to perform decalcification. After cutting the treated orbital tissue in paraffin, hematoxylin-eosin stain (H&E staining) (Chroma 1B, Schmid GmbH, Munster, Germany) was performed thereon. Then, the thicknesses of the eyelid and meibomian gland were measured, and the number of inflammatory cells was identified. The area of the adipose tissue around the optic nerve was identified. The process of performing the experiment is schematically shown in FIG. 1. FIG. 2 shows the results of comparing the orbit part of the SKG mouse in which the immune response was induced, with that of the control group not treated with zymosan A. The results of identifying the total eyelid thickness and meibomian gland thickness are shown in FIG. 3A, FIG. 3B, and FIG. 3C. The results of identifying the inflammatory cells around the orbit are shown in FIG. 3D and FIG. 3E.

As shown in FIG. 2, it was identified that blepharitis and exophthalmos occurred in both eyes in the SKG mouse group treated with zymosan A, compared to the control. As identified in FIG. 3A, it was identified that the total eyelid thickness and meibomian gland thickness were significantly increased in SKG mice treated with zymosan A compared to the control. As identified in FIG. 3B and FIG. 3C, it was identified that the thickness of the entire eyelid quantitatively increased by 1.15 times, and the thickness of the meibomian gland increased by 1.3 times. That is, it was identified that the thicknesses of the eyelid and meibomian gland increased significantly due to the administration of zymosan A.

Further, as identified in FIG. 3D and FIG. 3E, it was identified that invasion of inflammatory cells occurred significantly in the group treated with zymosan A compared to the control. Therefore, when zymosan A was administered into the SKG mouse, the thickness of the eyelid thereof increased as visible with the naked eye. It was identified histologically that the thicknesses of the eyelid and meibomian gland were significantly increased than that of the zymosan A non-treated group, resulting in blepharitis similar to that of Graves' ophthalmopathy.

2.2 Magnetic Resonance Imaging Analysis of Exophthalmos in Zymosan A-Treated SKG Mice

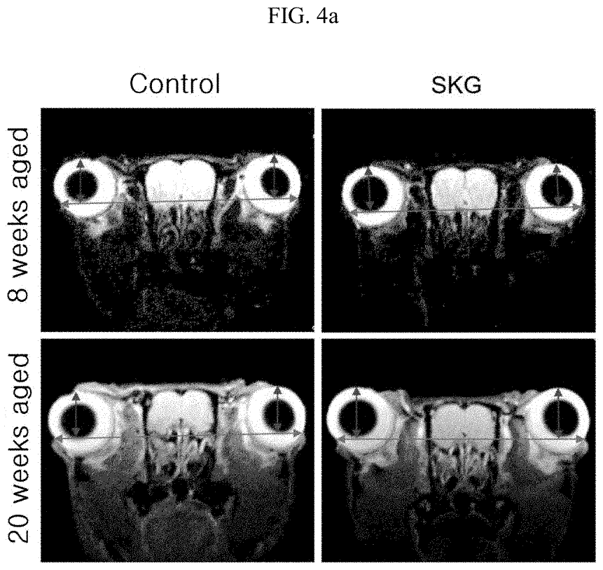

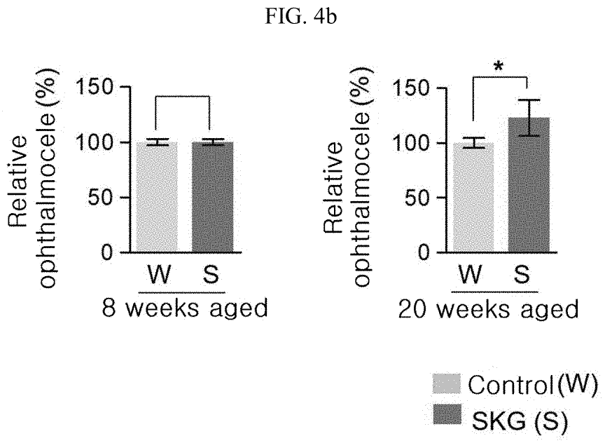

Magnetic Resonance Imaging of adult SKG mice was performed before zymosan A injection and 3 months after injection in order to identify changes in orbital tissue occurring in the mouse prepared in Example 1 above. In vivo MRI was performed with a horizontal bore 7T MRI scanner (Agilent Technologies Inc, USA). Mice were anesthetized using a 1 to 2% isoflurane-oxygen mix throughout the body magnetic resonance imaging (MRI) process. The mouse's head was placed in a 25 mm inner diameter quadrature MRI volume coil (PulseTeq Ltd, UK). A T2 weighted MRI image having a matrix size of 256×192 (100 μm planar resolution) and an average of 4 was taken under following conditions: Fast-spin-echo (FSE) sequence with a 4 second repetition time (TR); effective echo time (TE) of 60 seconds, a length of the echo train being 8, a RARE factor having a field of view (FOV) of 16, 26 mm×26 mm. 0.61 mm thick contiguous and coronal images including many parts of the eye and brain were obtained. The MR images of 24 contiguous regions from a rear eye portion of 0.4 mm with 94 μm planar resolution (perpendicular to a long axis of the eye and similar to a histological treatment direction) to a front eye portion were collected under following conditions: fast-spin-echo (FSE) chain with a repetition time (TR) of 1400 seconds; the effective echo time (TE) of 7.84 seconds, field of view (FOV) of 12 mm×12 mm, matrix size of 128×128 (94 μm planar resolution) and an average value of 24. Breathing and temperature were monitored throughout the MRI process while maintaining the body temperature at 37° C. using warm air (SA Instruments, USA). ImageJ (NIH) software was used not only to measure changes in orbital fat mass around the optic nerve in MR images, but also to interpret MR images. The magnetic resonance imaging result image is shown in FIG. 4.

As shown in FIG. 4, compared to the control, the volume of orbital adipose tissue in SKG mice was significantly increased before and after zymosan A injection. It was identified that inducing an intrinsic immune response by administering zymosan A to SKG mice could lead to blepharitis and exophthalmos, which are representative phenotypes of Graves' ophthalmopathy.

### Example 3. Exophthalmos Due to Increased Beige Fat Observed in Zymosan A-Treated SKG Mice

Histological analysis of the eyeballs of adult SKG mice was performed 3 months after administration of zymosan A to 8-week-old SKG mice to determine the mechanism of exophthalmos.

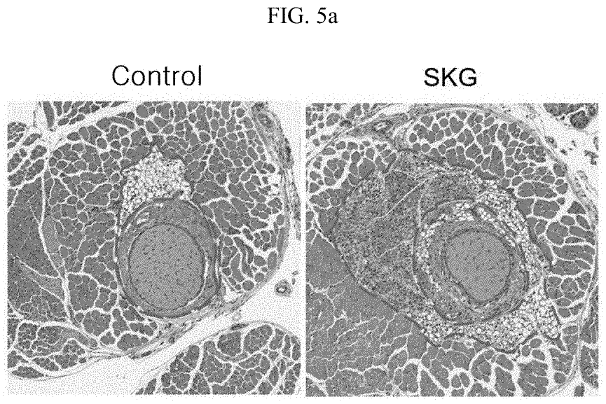

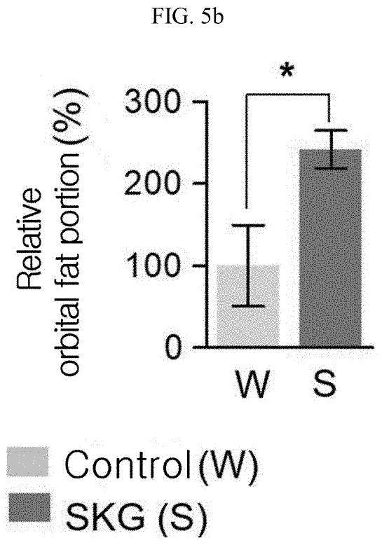

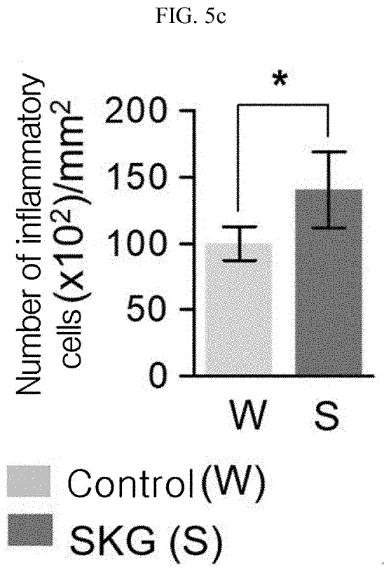

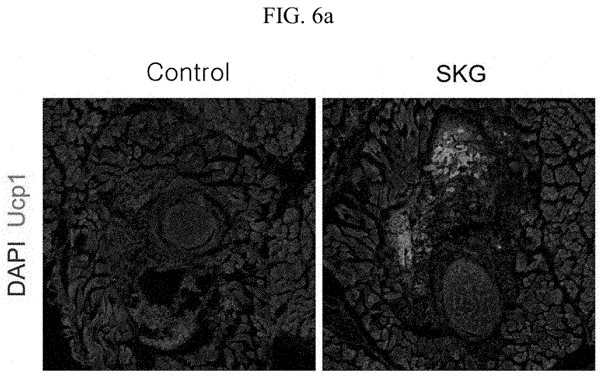

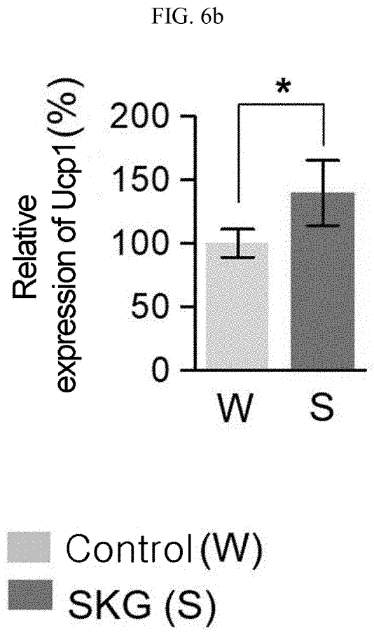

Immunohistochemical visualization of beige adipocytes was performed using a rabbit anti-mouse UCP-1 antibody (1:200, Alpha Diagnostic International, San Antonio, Tex., USA). The tissue was incubated overnight at 4° C. with the primary antibody. We visualized biotin-conjugated secondary antibodies (biotin goat-anti-rabbit, Santa Cruz Biotech Inc.) using commercially available ABC and DAB-kits (Vectastain ABC/DAB, Vector Labs, Burlingame, Calif., USA). Endogenous peroxidase activity was inhibited with 3% H₂O₂. The slides were counter-stained with hematoxylin for 90 seconds. The number of inflammatory cells was identified under 400 times magnification using a Nikon eclipse E1000 microscope, to identify adipose tissue and inflammatory cell infiltrate. It is widely known that the immune response via T cells is associated with adipose tissue metabolism, and autoimmune CD4+ T cells are produced in the thymus of SKG mice. Considering those facts, staining with uncoupling protein-1 (UCP-1) as a representative marker of beige adipocytes in orbital adipose tissue around the optic nerve was performed. Thus, the distribution of the UCP-1 positive adipocytes was determined by measuring the expression intensity using immunohistochemical staining to identify the distribution of beige fat. The orbital adipose tissue surrounding the optic nerve in the SKG mouse is shown in FIG. 5A. FIG. 5B shows the quantification graph thereof. FIG. 5C shows the quantification graph of the inflammatory cells infiltrating the orbit. The results of identifying the distribution of UCP-1 positive beige adipocytes are shown in FIG. 6A and FIG. 6B.

As identified in FIG. 5A and FIG. 5B, SKG mice treated with zymosan A had significantly increased orbital adipose tissue around the optic nerve by about 2.5 times when compared with the control. As identified in FIG. 5C, it was identified that the invasion of inflammatory cells in the orbital tissue of SKG mice occurred. Thus, it was identified that even in SKG mice treated with zymosan A, the orbital inflammatory response was a factor involved in the occurrence of exophthalmos in a similar manner to the case of real human Graves' ophthalmopathy.

As identified in FIG. 6A and FIG. 6B, based on a result of immunofluorescence staining using UCP-1 as a representative marker of beige fat, it could be identified that the adipose tissue significantly increased in SKG mice treated with zymosan A is beige fat, and therefore, when zymosan A was administered into SKG mice, the beige fat associated with the immune mechanism was significantly increased around the optic nerve.

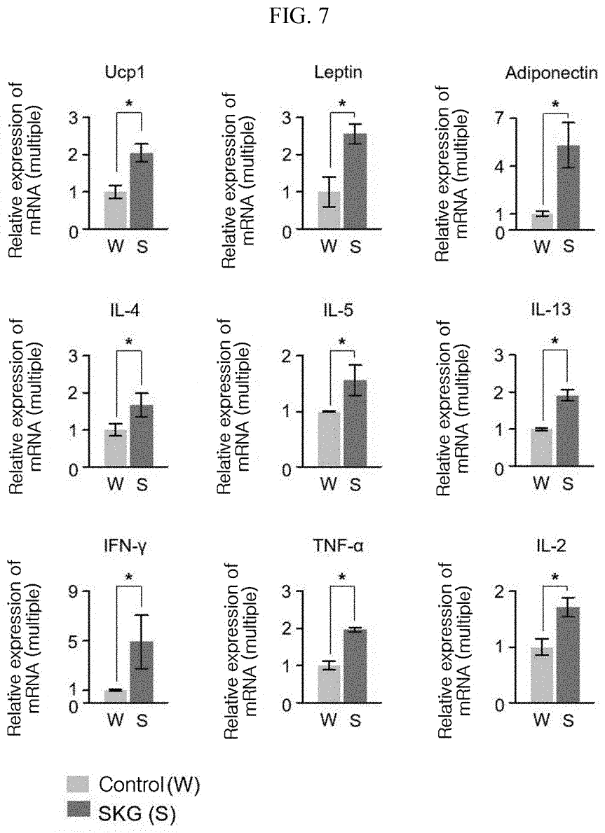

As identified in FIG. 7, when comparing SKG mice treated with zymosan A with the control, a significant increase in adipokines such as UCP-1 (uncoupling protein-1), adiponectin and leptin in the orbital tissue was identified. A significant increase in cytokines such as IL-4, IL-5 and IL-13 related to T cells could be identified together. Further, the findings of significantly increased inflammatory cytokines such as IFN-gamma, TNF-alpha and IL-2 were identified, thus suggesting that the increase in beige fat production was induced by these cytokines.

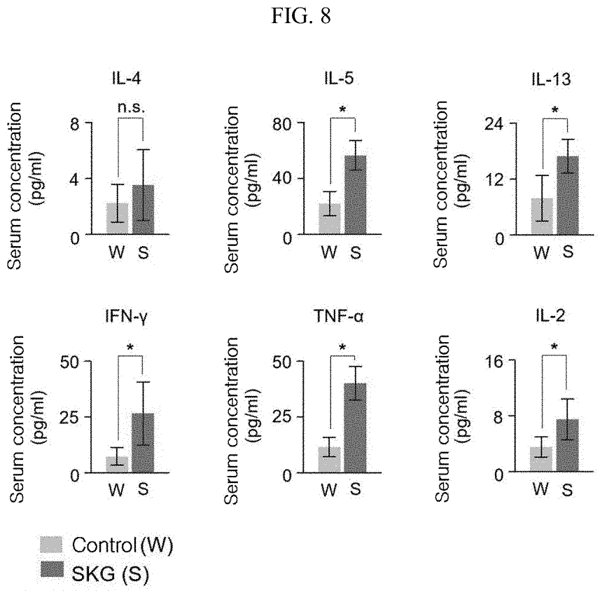

The concentration of cytokines in serum was measured in order to identify whether cytokines which increased in the orbital tissues were increased in the serum. As identified in FIG. 8, when comparing cytokines in serum of SKG mice treated with zymosan A to that of the control, increase in the concentrations of IL-4, IL-5, IL-13, IFN-γ, TNF-α and IL-2 in the serum of SKG mice treated with zymosan A was identified.

Taken together, the administration of zymosan A into the SKG mice causes increased inflammatory response and increased production of beige fat by an autoimmune reaction associated with T cells in orbital adipose tissue, which may lead to exophthalmos. Thus, it was identified that an animal model having phenotype similar to that of Graves' ophthalmopathy occurring in humans may be established.

1. A method for preparing a Graves' ophthalmopathy phenotype animal model, the method comprising administering zymosan A to a non-human subject.

2. The method of claim 1, wherein an administration amount of the zymosan A is in a range of 0.5 mg to 10 mg.

3. The method of claim 1, wherein the animal model has increased beige fat around an optic nerve.

4. The method of claim 1, wherein the Graves' ophthalmopathy phenotype is at least one selected from a group consisting of blepharitis and exophthalmos.

5. The method of claim 4, wherein the blepharitis and exophthalmos are accompanied by an increase in a thickness of an eyelid or an increase in a thickness of a meibomian gland.

6. The method of claim 1, wherein the Graves' ophthalmopathy phenotype animal model has increased expression of at least one adipokine selected from a group consisting of UCP-1 (uncoupling protein-1), leptin, adiponectin, IL-4, IL-5, IL-13, IL-2, IFN-γ and TNF-α in an orbital tissue thereof.

7. The method of claim 1, wherein the Graves' ophthalmopathy phenotype animal model has increased expression of at least one cytokine selected from a group consisting of IL-4, IL-5, IL-13, IFN-γ, TNF-α, and IL-2 in serum thereof.

8. The method of claim 1, wherein the animal model includes a mouse, rat, rabbit, dog, or guinea pig.

9. A Graves' ophthalmopathy phenotype animal model prepared by the method of claim 1.

10. A composition for preparing a Graves' ophthalmopathy phenotype animal model, the composition containing zymosan A as an active ingredient.

11. A method for screening a substance for alleviation or treatment of Graves' ophthalmopathy, the method comprising:
(a) administering a candidate substance for an alleviation or treatment agent for Graves' ophthalmopathy phenotype into the animal model of claim 9; and
(b) identifying an alleviation or treatment effect of the Graves' ophthalmopathy phenotype in the animal model into which the candidate substance is administered, compared to a control into which the candidate substance is not administered.
